(19) United States
(12) Patent Application Publication
(10) Pub. No.: US 2017/0112423 A1
(43) Pub. Date: Apr. 27, 2017

(54) System and Method for Stimulus Optimization Through Closed-Loop, Iterative Biological Sensor Feedback

(71) Applicants: Dylan Caponi, Manhattan Beach, CA (US); David Cardoza, Los Alamitos, CA (US)

(72) Inventors: Dylan Caponi, Manhattan Beach, CA (US); David Cardoza, Los Alamitos, CA (US)

(21) Appl. No.: 14/838,163

(22) Filed: Aug. 27, 2015

Related U.S. Application Data
(62) Division of application No. 13/855,780, filed on Apr. 3, 2013, now abandoned.
(60) Provisional application No. 61/619,910, filed on Apr. 3, 2012.

(51) Int. Cl. A61B 5/16; A61B 5/00

(52) U.S. Cl. CPC A61B 5/165; A61B 5/486

(57) ABSTRACT

A system and method for optimizing a stimulus, comprising a presentation device for presenting a stimulus to a subject, at least one biological sensor for measuring at least one biological parameter of a subject, and a computing device configured to determine a subject's emotional state in response to each stimulus, rank the stimuli in response to the subject's emotional state, use the higher-ranked stimuli to generate new stimuli, and present the new stimuli to the subject via the presentation device.

### CROSS-REFERENCE TO RELATED APPLICATIONS

The present application is a divisional application of U.S. application Ser. No. 13/855,780, filed Apr. 3, 2013, which claims the benefit of U.S. Provisional Patent Application No. 61/619,910, filed Apr. 3, 2012, which is hereby incorporated by reference in its entirety.

### BACKGROUND OF THE INVENTION

Field of the Invention

The present invention relates to media optimization methods, and in particular to a media optimization method based on closed-loop iterative biological sensor feedback evaluating the subject's emotional response.

Description of the Prior Art

Marketing and design professionals often use focus groups or test subjects to evaluate and optimize a typical viewer's emotional response to a particular product, logo, advertising jingle, or other stimuli, in an effort to create a stimulus with the maximum emotional impact. Currently, such design processes go through discrete stages—a prototype is developed, then a focus group evaluates the product, the design team analyzes the response of the focus group, creates another prototype based on that response, the focus group evaluates the new prototype, and so on.

One problem with this method is that focus group evaluations are by necessity highly subjective and not always detailed enough to be informative on exactly what needs to be changed to improve the product. In areas such as perfume design or music, it requires a lot of special training to even be able to apply the appropriate vocabulary, and it is often difficult to articulate just what causes a positive or negative response to a test stimulus. As a result, design teams often are unable to determine just what caused a negative response and what needs to be changed.

One way to solve this problem is by using a genetic algorithm to automatically generate new variations on a design, and using the user's feedback to select the “fittest” variations and use them to generate new ones. These methods are often used for cochlear implant fitting; for example, U.S. Pat. No. 6,879,608 to Wakefield et al. discloses such a system, in which a genetic algorithm operates to generate successive generations of multiple groups of values for a parameter subset, and patient feedback determines which half of the group of values are selected and then used to determine the values for the next generation. However, most of those systems and methods are subjective rather than objective, relying on conscious patient feedback. U.S. App. No. 2012/0290045 to Nicolai et al. discloses a similar system, in which one embodiment uses objective rather than subjective measurements; however, the objective measurement is relatively simple and only measures the action potential of the auditory nerve or various latency responses. Usually, such objective measurements are only used for very young or non-cooperative patients, since most patients have no reason to misrepresent the function of their cochlear implant fittings, are easily able to perceive which parameters sound better, and only need to answer simple questions about the loudness of the sound.

The reason such methods have not been used to determine aesthetic appreciation is because a complex stimulus such as a logo or a commercial jingle has many more parameters, and people are often unable to determine consciously what stimuli they prefer to what other stimuli. Also, some people may consciously or subconsciously misrepresent their preferences to please the experimenter, to preserve their social image, or for other reasons. As a result, automatic measurements of user perception have been inapplicable in the design and marketing world, and the design process continues to rely on conscious user reports, though they are significantly flawed. Furthermore, not every user is capable of making conscious reports; very young children, people with disabilities, or animals, are often incapable of expressing their preferences, though they may have them.

An automated method of measuring the user's aesthetic appreciation of complex stimuli and optimizing the creation of said complex stimuli is therefore needed.

### SUMMARY OF THE INVENTION

An object of the present invention is to optimize an interpreted emotional response of an individual or group by presenting them with an iteratively varied stimulus. The stimulus can be visual (such as a company logo), auditory (such as an advertising jingle), olfactory (such as a perfume), verbal (such as an advertising slogan), sexual, or any other type of stimulus that can be perceived by the individual or group.

Another object of the present invention is to use an optimization algorithm such as a genetic algorithm to iteratively generate new sets of stimuli based on the subject's interpreted emotional responses in order to create a stimulus or stimuli that achieves the desired emotional response.

A further object of the present invention is to use an optimization algorithm such as a genetic algorithm to iteratively generate new sets of stimuli based on the subject's interpreted emotional responses in order to create an emotional state in the subject.

The present invention provides a system and method for automatically optimizing a stimulus based on the emotional responses of a subject or subjects. In one aspect, the method of the present invention comprises presenting a subject with several initial stimuli one by one, using biological sensors to measure the subject's response to each stimulus, determining the subject's emotional state based on the output of the biological sensors, ranking the stimuli based on the subject's emotional response, selecting one or more highest-ranking stimuli, and using the highest-ranking stimuli to generate new stimuli to present to the subject under test. The steps of presenting the stimuli to the subject, measuring the subject's response, determining the subject's emotional state, ranking the stimuli, and selecting one or more highest-ranking stimuli, are then repeated until the desired level of optimization is achieved. In the preferred embodiment, this is done in real time, and the subject can perceive the stimulus optimizing itself in front of the subject in real time.

The stimuli can be any stimuli that can be appreciated aesthetically or emotionally by humans or animals. Any stimulus perceptible by the human senses can be optimized by the method of the present invention. The stimulus may be visual, auditory, olfactory, tactile, or gustatory, or any combination of the foregoing. For example, the stimuli can be visual art, music, perfume, product design, typography design, industrial design, taste design, slogan design, or any other stimuli that require aesthetic judgment and where it is often difficult to express exact reasons for liking or disliking a given variation on a stimulus. The stimuli may also be perceptible by the extended senses, such as balance, proprioception, nociception, kinesioception, thermoception, and so on. Stimuli perceptible by multiple senses may also be used, such as videos containing both visual and auditory information.

The biological sensor or sensors used in the method of the present invention can be an EEG, EKG, pneumograph, capnometer, electrodermograph, penile tumescence sensor, or any other sensor that can measure a biological property of the human or animal body.

The emotional state used in the analysis can be any state that can be reliably correlated to a biological sensor or sensors. For example, excitement, engagement, frustration, meditation, anxiety, happiness, sadness, anger, fear, sexual arousal, or any other emotion that can be reliably correlated to biological sensor data can be measured and used in the present invention.

The system of the present invention includes one or more biological sensors attached to the subject under test, a display device to display the stimuli to the subject, a computing device that analyzes the output of the biological sensor or sensors and uses the output to determine the subject's emotional state, and a computing device that selects the stimuli based on the output of the first computing device and implements an optimization algorithm to generate new stimuli based on the selected stimuli. The latter computing device may be the same as the former computing device.

### DETAILED DESCRIPTION OF THE SEVERAL VIEWS OF THE DRAWINGS

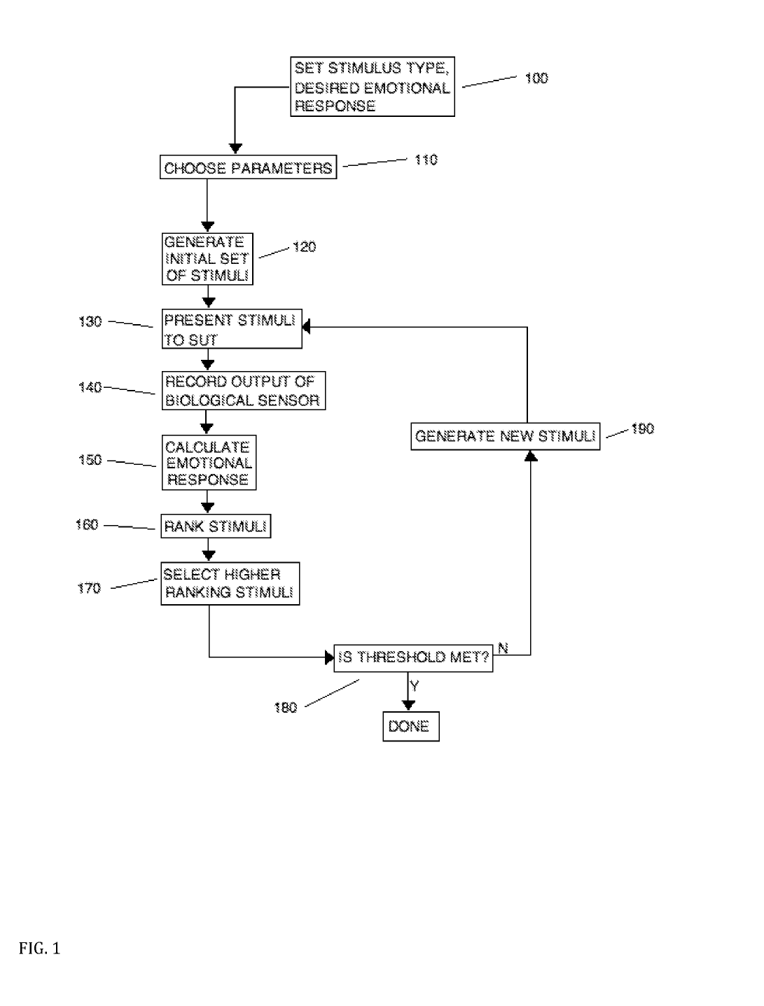

FIG. 1 shows a flowchart of an embodiment of the method of the present invention.

### DETAILED DESCRIPTION OF THE PREFERRED EMBODIMENT

FIG. 1 shows a flowchart of an embodiment of the method of the present invention. First, a stimulus to be optimized is chosen and a desired emotional or physiological response is set 100. The stimulus can be a product design, a company logo, an advertising jingle, a slogan, a scent, or any other stimulus that can be perceived by a human and that is intended to evoke an emotional response. The emotional response can be excitement, frustration, anger, happiness, engagement, sexual arousal, or any other emotion that can be reliably correlated with biological sensor data.

The presentation device by which a stimulus is presented to the subject could be a simple computer screen or speaker, or could be any other device that produces a stimulus perceptible to a human or animal subject. For visual stimuli, the presentation device could also be a TV, a projector, VR goggles, or direct stimulation of the visual cortex. For auditory stimuli, the presentation device could be headphones, speakers, or bone vibration drivers.

Other stimuli may require different presentation devices. For example, for somatic/tactile stimulation, the presentation device could be a vibrator, a rumble pack, electrical stimulation, electrical shock, a massage chair, or a climate control system. In the case of climate control, there could be more than one variable being controlled (e.g. humidity and temperature).

For gustatory stimulation, the user could be presented with small amounts of the food/drink/flavor to be sampled. Automatic generators of drinks such as cocktails, baby formula, or coffee already exist. It would be easy to connect the system of the present invention to such a generator to measure a user's response to the taste of each sample and to generate new samples based on the user's response.

Similarly, for olfactory stimulation, custom scent generators already exist. It would be easy to present the user with scent samples and to measure the user's response to each sample and use the responses to generate new scent samples.

For vestibular stimulation, such as self-balancing unicycles, skateboards, scooters, and so on, the system of the present invention could customize the responsiveness, top speed, and acceleration settings to minimize user frustration. In that case, the presentation device would simply be the vehicle itself.

The parameters of a stimulus are chosen by the experimenter 110, and their boundary values are set. For example, for a logo design, font type and size, color, and placement may be the variables—then, the boundary values can be the smallest and largest size of the font, the boundary values of the palette of colors to be chosen, and the extreme left, right, top, and bottom positions for placement. A set of random initial stimuli is generated based on those parameters 120.

The subject under test (SUT) is then outfitted with at least one biological sensor such as an EEG, EKG, pneumograph, capnometer, electrodermograph, and so on. The initial stimuli are then presented to the SUT one by one 130. As the SUT perceives each stimulus, the output of the biological sensor or sensors is recorded by the system and correlated with the appropriate stimulus 140. After exposure to each stimulus, a controllable amount of time passes, and then the system interprets data from the biological sensor or sensors and calculates a rating of how well the stimulus elicited the desired emotional or physiological response (fitness) 150. The stimuli are then ranked by their fitness 160, and one or more highest-ranking stimuli are selected 170.

If the threshold level of fitness has not yet been achieved 180, the optimization algorithm then operates on the highest-ranking stimuli and generates new stimuli from the highest-ranking stimuli 190. The new stimuli are then presented to the SUT 130. The steps of selecting the highest-ranking stimuli and generating new stimuli from the highest-ranking stimuli are then repeated until a threshold level-of-response is met. This threshold can be set ahead of time, or determined in real time by the experimenter or the SUT.

In an alternate embodiment of the present invention (not shown), the goal is not to produce an optimized stimulus but rather to produce a desired emotional state in the user—for example, to induce a meditative state. In that case, the steps of selecting the highest-ranking stimuli and generating new stimuli from the highest-ranking stimuli are repeated until the desired emotional state is maintained for the desired amount of time.

Any number of highest-ranking stimuli can be selected. The number of highest-ranking stimuli can also be varied as the optimization algorithm progresses.

The optimization algorithm may be a genetic algorithm. This is the preferred embodiment of the invention. The genetic algorithm is initialized with a set of random, but parameterized individual stimuli. Each individual stimulus is composed of one or more genes, a gene being a representation of one variable used to optimize the stimulus. In the initial set of stimuli, the genes are set randomly. After the initial set of stimuli is displayed to the SUT one by one, their fitness level is determined and a specified number of the highest-fitness stimuli proceed to a “mating” phase of the genetic algorithm. In that phase, stimuli swap random sections of genes in a process called crossover, or have their genes altered stochastically in a process called mutation. New stimuli generated by either one, or both, of these processes, are thus created and make up the next generation of stimuli. In one embodiment, a small portion of the previous generation that has the highest fitness is also allowed to pass into the next generation. The next generation of stimuli is then presented to the SUT, their fitness level is determined, and the highest-fitness stimuli then go through the “mating” phase again. This is repeated until the desired fitness level is reached. As a result, a SUT can watch a logo or a product design improve itself in real time in front of them.

The “parents” of each individual (i.e. the two stimuli whose genes are swapped to create new stimuli) can be selected randomly, or the probability of each stimulus being selected to be a parent can depend on its fitness level.

The genetic algorithm may be tailored in several different ways. For example, the number and average span of crossovers, the mutation probability, the selection type, the highest-fitness group size, and the initial population size are all parameters that can be varied depending on the problem at hand. The algorithm may also adjust these parameters dynamically as the optimization process advances.

The applications of the present invention are numerous, and though many are below-listed, many are omitted due to their similarity in terms of product and goal to those already listed. Any product that attempts to elicit an emotional or physiological response by appealing to any of the five traditional senses (or extended senses) to optimize the experience or absence of a currently known (or developed in the future) interpreted emotion or defined physiological state by the use of any biological sensor, can benefit from the use of this invention. Some sample applications include:

a. jingle design,

b. video editing and segment duration,

c. organizing advertisement video sequences, selecting video sequences,

d. logo design (font type, style, size, color etc.), company name design

e. word design for visual and auditory aesthetics

f. designing smells

g. designing tastes

h. casting actor combinations

i. avatar aesthetic design

j. cartoon character aesthetic design

k. web page design

l. speech design

m. aesthetic appearances of any product (clothing, electronics, car shapes, accessories),

n. color template design

o. graphical user interface (GUI) design and physical interface design (minimizing frustration)

p. store floor plan layout,

q. physical sensations

r. environmental design

s. virtual/real indoor/outdoor lighting colors

t. magazine covers

u. to optimize a specific physiological state or biological response in a focus group or individual.

We also note that information gleaned from this method can provide valuable statistical data regarding the emotional state of people with regard to stimuli presented. This can allow marketing groups to generate a general understanding (if one exists) of how an individual, or groups of similar individuals, will respond to marketing media. Furthermore, this can be used to understand how biases brought on by cognitive interactions can both positively and negatively influence media design.

The biological sensors used for the present method can be any sensors that measure a biological phenomenon that can be correlated to an emotion. Some sample sensors that can be used are EEG, EKG, pneumograph (respiration rate), capnometer (CO2 output), or electrodermograph (skin conductance), penile tumescence sensor, pulse oximeter. Other sensors may also be used.

The emotional responses that are evaluated by the present method are any emotions that can be interpreted by biological sensors. For example, the Emotiv EPOC consumer EEG device can measure and rate a SUT's excitement, engagement, frustration, and meditation. Other emotions may also be evaluated by other sensors or by other evaluation systems. For example, an electrodermograph measures skin conductance, which correlates to surprise, arousal, worry, or cognitive activity. A capnometer measures CO2 output, which correlates to stress or anxiety. The vagal tone (the relationship between breathing and heart rate) correlates to happiness, sadness, anger, and fear. Many other emotions have been interpreted by a range of biological sensors and documented in psychological studies.

Some potential shortcomings of the present invention are the large number of stimuli required to optimize a complex stimulus such as a logo or a jingle, and subject exhaustion to the stimuli. The first problem can be alleviated by limiting the number of variables that can be controlled by the algorithm, thus reducing its search space. The second problem, subject exhaustion, arises when a subject loses interest in the stimuli, or becomes fatigued, after being shown hundreds of pictures or other stimuli. Two ways to counter this problem are limiting the length of stimuli exposure sessions and rating stimuli based on a moving average of the recent history of fitness values. Another shortcoming of the present invention is that if the stimuli are not effective enough to engage the subject, the effect of the stimuli will be less than the noise of the subject's daydreaming or neutral disposition. The stimuli optimized by the present invention must be effective enough to engage the SUT and the SUT has to be attentive to the stimuli.

1. A system, comprising:
at least one biological sensor that can sense a biological parameter of a human or animal subject;
a presentation device for presenting at least one stimulus to the subject;
a computing device configured to perform the following:
determining an emotional state of the subject based on the output of the at least one biological sensor when a stimulus is presented to the subject;
ranking the stimuli presented to the subject based on the emotional state;
generating new stimuli based on the higher-ranked stimuli;
connecting to the presentation device to present the new stimuli to the subject;
determining whether any of the stimuli meet a threshold of acceptability;
repeating the determining, ranking, generating, and connecting steps until the threshold of acceptability is reached.

2. The system of claim 1, where the at least one biological sensor is selected from the group consisting of:
an electrocardiograph;
a pneumograph;
a capnometer;
an electrodermograph;
a penile tumescence sensor;
a pulse oximeter;
a papillary response sensor;
a facial electromyograph;
and combinations of the foregoing.

3. The system of claim 14, where the stimuli are selected from the group consisting of:
visual stimuli;
auditory stimuli;
olfactory stimuli;
tactile stimuli;
gustatory stimuli;
thermoceptive stimuli;
proprioceptive stimuli;
nociceptive stimuli;
equilibrioceptive stimuli;
kinesthesioceptive stimuli;
sexual stimuli;
and combinations of the foregoing.

4. The system of claim 1, wherein the computing device generates new stimuli based on a genetic algorithm.

5. The system of claim 1, wherein the computing device generates new stimuli by performing the following steps:
assigning a variable to each parameter of the stimulus;
swapping random sections of variable between at least one stimulus and at least one other stimulus;
creating new stimuli based on the swapped variables.

6. The system of claim 5, wherein the computing device also stochastically alters at least one variable in at least one stimulus.

7. The system of claim 1, wherein the presentation device is one of the following: a computer screen, a speaker.
